(19) United States
(12) Patent Application Publication
(10) Pub. No.: US 2025/0387324 A1
(43) Pub. Date: Dec. 25, 2025

(54) IN SITU FORMING IMPLANTS AND MICROPARTICLES FOR INTRAARTICULAR DRUG DELIVERY

(71) Applicant: Mississippi State University, Starkville, MS (US)

(72) Inventor: Stephen H. Elder, Starkville, MS (US)

(21) Appl. No.: 19/240,873

(22) Filed: Jun. 17, 2025

Related U.S. Application Data
(60) Provisional application No. 63/662,927, filed on Jun. 21, 2024.

(51) Int. Cl. A61K 9/00; A61K 31/704; A61K 47/10; A61K 47/22; A61K 47/34

(52) U.S. Cl. CPC A61K 9/0024; A61K 31/704; A61K 47/10; A61K 47/22; A61K 47/34

(57) ABSTRACT

Implants and microparticles for intraarticular drug delivery are provided herein. The implants and microparticles can be formed in situ in a joint for sustained drug delivery, for example for the treatment of osteoarthritis. Methods of treating osteoarthritis are also provided.

### RELATED APPLICATIONS

This application claims priority from U.S. Provisional Application Ser. No. 63/662,927, filed Jun. 21, 2024, the entire disclosure of which is incorporated herein by this reference.

### GOVERNMENT INTEREST

This invention was made with government support under 5R25GM123920-03, T35OD010432 awarded by National Institutes of Health. The government has certain rights in the invention.

### TECHNICAL FIELD

The present invention generally relates to the field of drug delivery. More specifically, the present invention relates to intraarticular drug delivery.

### BACKGROUND

Osteoarthritis drugs injected into a joint such as the knee are cleared from the joint very rapidly due to efficient lymphatic drainage. However, effectiveness of some types of drugs relies on long-term exposure. Aspects of the present invention include a system to release a drug steadily for many weeks to several months after injection into a joint. In an aspect, the drug under investigation is punicalagin, a polyphenol found in pomegranates.

Patients with osteoarthritis may receive intraarticular injections of hyaluronic acid (viscosupplementation) or corticosteroids to relieve symptoms. Neither of these drugs modify the course of the disease. That is, they do not slow the progressive erosion of cartilage. Punicalagin, for example, has the potential to halt or slow cartilage degeneration by inactivating the main enzyme responsible for cartilage destruction and by decreasing production of such enzymes, as well as other inflammatory mediators. The chondroprotective effects of punicalagin, like many other drugs, diminish rapidly once it is cleared from joint. Therefore, its delivery needs to be sustained as long as possible.

### SUMMARY

An aspect of the present disclosure is a method of forming an implant, the method comprising: providing a mixture comprising a water-miscible organic solvent; a water-immiscible organic solvent; a biodegradable polyester comprising poly(lactic-co-glycolic acid), polycaprolactone, or a combination thereof; and a disease-modifying osteoarthritis drug; and contacting the mixture with an aqueous environment to form a precipitate from the mixture to provide the implant.

Another aspect is an implant for intraarticular drug delivery.

Another aspect is a method of forming a plurality of microparticles, the method comprising: combining a biodegradable polyester comprising poly(lactic-co-glycolic acid), polycaprolactone, or a combination thereof; and a disease-modifying osteoarthritis drug, in a solvent to provide a first phase; combining the first phase with a second phase comprising an oil to provide an emulsion, wherein the first phase is a minor phase and the second phase is a major phase; and contacting the emulsion with an aqueous environment to precipitate the plurality of microparticles from the emulsion.

Another aspect is a plurality of microparticles comprising a biodegradable polyester comprising poly(lactic-co-glycolic acid), polycaprolactone, or a combination thereof and a disease-modifying osteoarthritis drug.

Another aspect is a method of treating osteoarthritis by intraarticular injection of punicalagin.

### BRIEF DESCRIPTION OF THE DRAWINGS

FIG. 1A shows effect of punicalagin (PCG) on production of IL-1β by human THP-1 macrophages (mean±standard deviation, n=4). Asterisks indicate statistically significant differences (p<0.05).

FIG. 1B shows effect of PCG on production of prostaglandin E2 (PGE2) by human THP-1 macrophages (mean±standard deviation, n=4). Asterisks indicate statistically significant differences.

FIG. 2 shows PCG release from PLGA in situ forming implants (ISI) (75:25 lactide: glycolide, 20% w/w) formed using N-methyl-2-pyrrolidone (NMP) or benzyl alcohol (BA) as the only solvent.

FIG. 3 shows effect of NMP/benzyl benzoate (BB) ratio on PCG release from PLGA ISIs (75:25 lactide: glycolide, 20% w/w).

FIG. 4 shows effect of polymer type on release of PCG from ISIs made using NMP/BB 70/30 as solvents. Polymer concentration was 20%, 75:25 and 50:50 refer to lactide: glycolide ratio.

FIG. 5 shows effect of PLGA concentration on rate of PCG release from ISIs made using NMP/BB 70/30 as solvents. PLGA lactide: glycolide ratio was 75:25.

FIG. 6 shows PCG release from ISI made using PLGA (75:25) at 20% w/w and NMP/BA/BB 20/40/40 as solvents

FIG. 7A shows inhibition of type 2 collagenase by PCG (mean±standard deviation) where PCG was added directly to collagenase solution. Asterisks indicate statistically significant differences compared to the control cartilage disks collected at the same time (p<0.05).

FIG. 7B shows inhibition of type 2 collagenase by PCG (mean±standard deviation) where PCG was released from PLGA ISIs. Asterisks indicate statistically significant differences compared to the control cartilage disks collected at the same time (p<0.05).

FIG. 8 shows a liquid chromatography with tandem mass spectrometry (LC-MS/MS) chromatogram of Day 3-Day 7 sample of water into which PCG had released from a PLGA in situ forming implant.

FIG. 9 shows injection forces for ISI made using PLGA (75:25) at 20% w/w and NMP/BA/BB 20/40/40 as solvents (mean±standard deviation of three trials).

FIG. 10 shows viability of primary porcine chondrocytes exposed to PLGA ISIs (75:25, 20% w/w) made using NMP/BA/BB 20/40/40 as solvents (mean±standard deviation). Asterisk indicates statistically significant difference with respect to the non-exposed control group.

### DETAILED DESCRIPTION

The details of one or more aspects of the presently disclosed subject matter are set forth in this document. Modifications to aspects described in this document, and other aspects, will be evident to those of ordinary skill in the art after a study of the information provided in this document. The information provided in this document, and particularly the specific details of the described exemplary aspects, is provided primarily for clearness of understanding and no unnecessary limitations are to be understood therefrom. In case of conflict, the specification of this document, including definitions, will control.

While the terms used herein are believed to be well understood by those of ordinary skill in the art, certain definitions are set forth to facilitate explanation of the presently-disclosed subject matter. Unless defined otherwise, all technical and scientific terms used herein have the same meaning as is commonly understood by one of skill in the art to which the invention(s) belong. Where reference is made to a URL or other such identifier or address, it is understood that such identifiers can change and particular information on the internet can come and go, but equivalent information can be found by searching the internet. Reference thereto evidences the availability and public dissemination of such information.

Although any methods, devices, and materials similar or equivalent to those described herein can be used in the practice or testing of the presently-disclosed subject matter, representative methods, devices, and materials are described herein.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise. When open-ended terms such as “including” or ‘including, but not limited to” are used, there may be other non-enumerated members of a list that would be suitable for the making, using or sale of any aspect thereof.

Following long-standing patent law convention, the terms “a,” “an,” and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some aspects ±20%, in some aspects ±10%, in some aspects ±5%, in some aspects ±1%, in some aspects ±0.5%, and in some aspects ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

Throughout this application, various publications may be referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

The global prevalence of osteoarthritis (OA) ranges from 2090 to 6128 cases per 100,000 population, with the highest burden of disease found in the United States.¹ The prevalence of disease is also rising; for example, it increased by 23.2% in the U.S. between 1990 and 2017.1 The burden of OA is associated with pain, stiffness, decreased range of motion, and swelling which restricts activity and diminishes quality of life. In fact, OA is among the leading causes of years lived with disability,² and the medical cost in some developed countries may be as high as 2.5% of GDP.³ Therefore, there is strong interest in disease-modifying OA drugs (DMOADs) that can be injected intraarticularly. The benefit of this localized delivery is that it maximizes drug activity at the target location, while minimizing exposure of other organs and the risk of unwanted side effects.⁴ Furthermore, orthopedists are trained to perform intraarticular injections for viscosupplementation, and the procedure is safe with low risk of infection.^{5,6} However, because lymphatic drainage of intraarticularly injected drugs is so efficient, the drug dwell time is quite short.

There is no cure for OA, and the two most common intraarticular treatments, corticosteroids and hyaluronic acid, are for pain relief and increased joint range of motion. Due to the efficient drainage of the joint, the development of intraarticular depots for long-lasting drug release is a difficult challenge. Moreover, a disease-modifying osteoarthritis drug (DMOAD) that can effectively manage osteoarthritis has yet to be identified. Thus, there is an urgent, unmet need for disease-modifying OA drugs (DMOADs) that would actually inhibit disease progression. Punicalagin (PCG) has been identified as a promising DMOAD candidate.

The present inventor discovered injectable, in situ forming implants (ISIs) that create depots supporting the sustained release of punicalagin, a promising DMOAD. In vitro experiments demonstrated punicalagin's ability to suppress production of interleukin-1β and prostaglandin E2, confirming its chondroprotective properties. Regarding the entrapment of punicalagin, it was demonstrated by LC-MS/MS to be stable within a biodegradable polyester such as poly(lactic-co-glycolic acid) (PLGA) or polycaprolactone (PCL) in situ forming implants for several weeks and capable of inhibiting collagenase upon release. In vitro punicalagin release kinetics were tunable through variation of solvent, PLGA lactide: glycolide ratio, and polymer concentration, and an optimized formulation supported release for approximately 90 days. The injection force of this formulation steadily increased with plunger advancement and higher rates of advancement were associated with greater forces. Although the optimal formulation was highly cytotoxic to primary chondrocytes if cells were exposed immediately or shortly after implant formation, upwards of 65% survival was achieved when the implants were first allowed to undergo a 24-72 h period of phase inversion prior to cell exposure. Aspects of the present invention include a polyester-based in situ forming implant for the controlled release of punicalagin. With modification to address cytotoxicity, such an implant may be suitable as an intraarticular therapy for OA.

The present inventor also sought to create a biodegradable polyester-based, in situ forming delivery system capable of ultralong, intraarticular PCG delivery. Such a system may be capable of semiannual injections that could be given prophylactically to patients at high risk of developing OA or to slow disease progression in patients with early-stage OA. PCG is the major polyphenol present in pomegranate. It plays regulatory roles in multiple signaling pathways involved in the inflammatory process and also inactivates MMP-13, the enzyme primarily responsible for destruction of cartilage collagen in OA.

The intraarticular route of administration is a novel one for in situ forming microparticles. In one aspect of the present invention, fabrication of in situ-forming microparticles (ISMs) begins by dissolving a biodegradable polyester comprising PLGA, PCL, or a combination thereof and a drug in a solvent such as N-methyl pyrrolidine (NMP) to create an internal phase, which is emulsified into a biocompatible external oily phase such as sesame oil. When the emulsion is injected into an aqueous environment, solvent diffuses out of the droplets, and the PLGA, PCL, or combination thereof and drug precipitate and form microparticles. The entrapped drug may diffuse out of the microparticles and also release as the polymer hydrolyses.

Aspects of the present invention including an in situ forming implant may also address the need for improved delivery depots for OA. Such implants form through a process of controlled polymer transformation from a liquid phase to a solid phase. When a solvent/polymer/drug solution is exposed to water, the efflux and influx of solvent and water, respectively, cause the polymer concentration to increase until the polymer's solubility limit is exceeded and it undergoes phase inversion, becoming a solid. Use of weaker solvents with low water miscibility allows for slow phase inversion and the formation of uniformly dense structures that exhibit zero-order drug release kinetics.⁷

This disclosure describes an in situ forming implant for ultralong sustained delivery of punicalagin (PCG), a candidate DMOAD. PCG (MW 1084.71) is the major polyphenol present in pomegranate (Punica granatum L.), and it contributes to the anti-inflammatory properties of this fruit.⁸ Orally administered pomegranate fruit extract has been shown to lessen the severity of induced OA in rats and rabbits,^{9,10} and daily intraperitoneal doses of PCG significantly reduced paw edema in an adjuvant-induced arthritis rat model.¹¹ The inventors have shown that semi-weekly, intraarticular injections of PCG seemed to result in less overall erosion of cartilage compared to a saline control in a monoiodoacetate-induced model of OA in rats.¹²

PCG is of particular interest because it targets cartilage degeneration and synovium inflammation. The present inventor has shown that PCG can interact with collagenase in vitro and inhibit its enzymatic activity.¹² It has been shown to exert a similar inhibitory effect on matrix metalloproteinase-13 mediated degradation of type II collagen, as well as on interleukin-1 beta-induced release of proteoglycan.¹¹ PCG can also prevent the degeneration of type II collagen by binding directly to it, which may block access of destructive enzymes to the fiber. For example, PCG was shown to bind non-covalently to collagen type II with high affinity via multiple hydrogen bonds (punicalagin has 17 hydroxyl groups) and x-x and electrostatic interactions.¹¹ In addition to inhibiting cartilage degeneration, PCG can also suppress synovium inflammation. For example, PCG suppressed lipopolysaccharide-stimulated production of the inflammatory mediators nitric oxide (NO), prostaglandin E2 (PGE2), and IL-6 cytokine by murine monocyte/macrophage-like cells in a dose-dependent manner.⁸

Accordingly, an aspect of the present disclosure is a method of forming an implant. The method comprises providing a mixture comprising a water-miscible organic solvent; a water-immiscible organic solvent; a biodegradable polyester comprising poly(lactic-co-glycolic acid), polycaprolactone, or a combination thereof; and a disease-modifying osteoarthritis drug.

The water-miscible and water-immiscible organic solvents are not particularly restricted provided that they meet the water miscibility criteria, are miscible with each other, and at least one of the water-miscible and water-immiscible organic solvents is capable of dissolving the a biodegradable polyester comprising poly(lactic-co-glycolic acid), polycaprolactone, or a combination thereof and a disease-modifying osteoarthritis drug. As used herein, “water-miscible” refers to a solvent which is capable of forming a homogenous mixture with water (i.e., is completely soluble in water) all proportions at a given temperature and pressure. The water-miscible solvent and water mix completely and uniformly without phase separation. In some aspects, the water miscible solvent can have a water solubility of greater than or equal to 10 grams per 100 milliliters of water at 20° C. Conversely, “water-immiscible” refers to a solvent which is incapable of mixing with water or an aqueous solution to form a single liquid phase under standard conditions. In mixtures with water, the solvent and water form distinct phases due to their low mutual solubility. In some aspects, the water-immiscible solvent can have a water solubility of less than 10 grams per 100 milliliters of water at 20° C., for example less than 5 grams per 100 milliliters of water, or less than 1 gram per 100 milliliters of water.

Exemplary water-miscible solvents can include, but are not limited to, methanol, ethanol, isopropanol, acetone, acetonitrile, dimethyl sulfoxide (DMSO), N-methyl-2-pyrrolidone (NMP), tetrahydrofuran (THF), ethylene glycol, propylene glycol, diethylene glycol, triethylene glycol, 1,4-dioxane, N,N-dimethylformamide (DMF), and the like, or a combination comprising at least one of the foregoing. In a specific aspect, the water-miscible solvent can comprise N-methyl-2-pyrrolidone (NMP).

Exemplary water-immiscible solvents can include, but are not limited to, benzyl alcohol, benzyl benzoate, toluene, xylene, hexane, heptane, cyclohexane, dichloromethane, chloroform, diethyl ether, ethyl acetate, butyl acetate, methyl tert-butyl ether, and the like, or a combination comprising at least one of the foregoing. In a specific aspect, the water-immiscible solvent can comprise benzyl alcohol, benzyl benzoate, or a combination thereof. In another specific aspect, the water-immiscible solvent comprises benzyl alcohol and benzyl benzoate.

In an aspect, the water-miscible solvent can comprise N-methyl-2-pyrrolidone and the water-immiscible solvent comprises benzyl alcohol and benzyl benzoate. The weight ratio of N-methyl-2-pyrrolidone:benzyl benzoate:benzyl alcohol can be 10-30:30-50:30-50, for example 15-25:35-45:35:45. In a specific aspect, the weight ratio of N-methyl-2-pyrrolidone:benzyl benzoate:benzyl alcohol can be 20:40:40.

Poly(lactic-co-glycolic acid) (PLGA) is a biodegradable and biocompatible copolymer composed of lactic acid and glycolic acid monomer units. PLGA is synthesized through the random ring-opening copolymerization of lactide and glycolide monomers, resulting in a polymer backbone characterized by ester linkages. The molar ratio of lactic acid to glycolic acid can be varied to control the polymer's physical and mechanical properties, such as crystallinity, glass transition temperature, degradation rate, and hydrophilicity. PLGA undergoes hydrolytic degradation into its monomeric constituents, which are naturally metabolized in vivo to carbon dioxide and water.

The poly(lactic-co-glycolic acid) for use in the present disclosure can have a lactic acid: glycolic acid molar ratio of 15:85 to 85:15, or 20:80 to 80:20, or 25:75 to 75:25, or 35:65 to 65:35, or 40:60 to 60:40, or 45:55 to 55:45, or 50:50. In a specific aspect, the poly(lactic-co-glycolic acid) can have a lactic acid: glycolic acid molar ratio of 60:40 to 80:20, or 70:30 to 80:20, or 72:28 to 78:22, or 75:25.

In some aspects, the biodegradable polyester can comprise polycaprolactone (PCL). Polycaprolactone is a biodegradable, semicrystalline aliphatic polyester synthesized via the ring-opening polymerization of E-caprolactone monomers. Due to its relatively slow degradation rate and mechanical properties, PCL can be advantageous for use in medical applications, including drug delivery systems and biodegradable implants. Its chemical structure generally consists of repeating units derived from caprolactone, with an ester linkage in the polymer backbone, which facilitates hydrolytic and enzymatic degradation under physiological conditions.

The biodegradable polyester comprising poly(lactic-co-glycolic acid), polycaprolactone, or a combination thereof can be present in the mixture in an amount of 15 to 30 weight percent, based on the total weight of the mixture. Within this range, the biodegradable polyester comprising poly(lactic-co-glycolic acid), polycaprolactone, or a combination thereof can be present in an amount of 17 to 28 weight percent, or 20 to 25 weight percent.

The disease-modifying osteoarthritis drug for use in the present disclosure is defined as a therapeutic agent that, when administered to a subject, is capable of altering the underlying pathophysiology of osteoarthritis (OA) by modifying the disease process, as opposed to merely alleviating symptoms such as pain or inflammation. A DMOAD achieves one or more of the following: slowing or halting cartilage degradation, promoting cartilage repair or regeneration, modifying subchondral bone structure, or affecting synovial tissue inflammation, thereby reducing disease progression and improving joint function.

Examples of agents considered DMOADs include, but are not limited to, matrix metalloproteinase (MMP) inhibitors, aggrecanase inhibitors, inhibitors of inflammatory cytokines such as interleukin-1 (IL-1) and tumor necrosis factor-alpha (TNF-α), growth factors that promote cartilage repair such as transforming growth factor-beta (TGF-β) and bone morphogenetic proteins (BMPs), fibroblast growth factor (FGF), hyaluronic acid derivatives, bisphosphonates, and small molecule compounds or biologics that stimulate chondrocyte anabolic activity or inhibit catabolic pathways involved in cartilage degradation. Additionally, certain monoclonal antibodies, peptides, and gene therapy approaches targeting molecular pathways implicated in osteoarthritis pathogenesis may also be classified as DMOADs. In an aspect, the DMOAD may include kartogenin.

The present inventor has further discovered that certain polyphenols may exhibit certain qualities of a DMOAD, likely due to their ability to inhibit inflammatory mediators, reduce oxidative stress, and modulate signaling pathways involved in cartilage degradation and joint inflammation (without wishing to be bound by theory). In a specific aspect, the DMOAD of the present disclosure can be a polyphenol. In some aspects, the DMOAD of the present disclosure can comprise castalagin, castalin, casuarictin, chebulagic acid, chebulinic acid, curcumin, gallic acid, gemin D, grandinin, hesperidin, pedunculagin, proanthocyanidin, punicalagin, punicalin, quercetin, resveratrol, roburin A, strictinin, tellimagrandin I, tellimagrandin II, terflavin A, terflavin B, tergallagin, and vescalagin, and the like, or a combination thereof. In a specific aspect, the DMOAD can comprise punicalagin.

The DMOAD can be present in the mixture in an amount effective to provide a concentration of DMOAD in the final implant suitable to exhibit cytokine suppression. The skilled person understands that variations in cytokines may require differing concentrations to achieve the desired therapeutic effect. Guided by the present disclosure, the skilled person would be able to select a suitable DMOAD concentration in the implant based on the cytokine of interest. In some aspects, the concentration of DMOAD in the final implant can be greater than 0 to 20 weight percent, or greater than 0 to 15 weight percent, or greater than 0 to 10 weight percent, or greater than 0 to 5 weight percent, or 0.1 to 10 weight percent, or I to 10 weight percent, or 0.1 to 5 weight percent, or 1 to 5 weight percent, or 2 to 4 weight percent, each based on the total weight of the final implant.

The mixture formed from the water-miscible organic solvent, the water-immiscible organic solvent, the biodegradable polyester, and the disease-modifying osteoarthritis drug is preferably a homogenous mixture. As used herein, the term “homogenous mixture” refers to a physical system in which the components of the mixture are uniformly distributed at the molecular level, resulting in a single-phase solution without visible separation or phase boundaries. In some aspects, the homogenous mixture is a clear, single-phase liquid.

The mixture is also preferably injectable. An injectable mixture refers to a formulation, that is suitable for administration via injection through a needle into a biological system. Such a mixture is typically sterile, free of particulate matter that could obstruct the needle, and possesses appropriate viscosity to allow for smooth passage through standard injection devices without causing undue irritation or damage to tissues. In some aspects, the injectable mixture is suitable for direct administration into a joint space by injection. The force required to inject the mixture through a 21G needle at a rate of up to 0.5 mm/s can be less than or equal to 45N. In some aspects, the force required can be 1 to 30 N, or 5 to 15 N.

The method of forming the implant further comprises contacting the mixture with an aqueous environment to form a precipitate from the mixture to provide the implant. The aqueous environment can be a biological environment, for example an in vivo biological environment. The precipitate (and thus the implant) comprises the biodegradable polyester and the disease-modifying osteoarthritis drug. In some aspects the contacting comprises injecting the mixture intraarticularly, and the implant is formed in situ in a joint.

Advantageously, the implant comprising the biodegradable polyester and the disease-modifying osteoarthritis drug embedded therein (e.g., in a joint) can provide a prolonged and sustained release of the drug from the implant. In some aspects, the implant does not exhibit a burst release profile. For example, in an aspect, less than 25% of the disease-modifying osteoarthritis drug in the implant is released from the implant after 15 days in an aqueous environment. In some aspects, up to 30 days, or up to 60 days, or up to 90 days are needed for the entirety of the loaded drug to be released.

An implant for intraarticular drug delivery made by the method described herein represents another aspect of the present disclosure. An intraarticular implant refers to a medical device or composition designed for direct placement or injection into a joint space to provide therapeutic benefits, including mechanical support, lubrication, drug delivery, or tissue regeneration. These implants can be solid, semi-solid, or gel-like in nature and may be composed of biocompatible or biodegradable materials. The intraarticular implant is capable of releasing the DMOAD over a sustained period, effective to modify joint mechanics, or promote cartilage repair and regeneration. The implant's properties including size, shape, viscoelasticity, and degradation profile can be tailored to ensure compatibility with the joint environment and to minimize adverse tissue reactions.

In an aspect, an implant can comprise a biodegradable polyester comprising poly(lactic-co-glycolic acid), polycaprolactone, or a combination thereof and a disease-modifying osteoarthritis drug contained therein. In an aspect, the disease-modifying osteoarthritis drug comprises an ellagitannin, such as punicalagin. In some aspects, the disease-modifying osteoarthritis drug can be present in the implant in an amount of greater than 0 to 10 weight percent, or greater than 0 to 5 weight percent, or 0.1 to 10 weight percent, or 1 to 10 weight percent, or 0.1 to 5 weight percent, or 1 to 5 weight percent, or 2 to 4 weight percent, each based on the total weight of the implant.

Another aspect of the present disclosure is a method of forming a plurality of microparticles. The microparticles are capable of providing a similar function as the implants already described herein, and may provide additional benefits such as reduced cytotoxicity. Accordingly, the method comprises combining a biodegradable polyester comprising poly(lactic-co-glycolic acid), polycaprolactone, or a combination thereof and a disease-modifying osteoarthritis drug in a solvent to provide a first phase. The biodegradable polyester and a disease-modifying osteoarthritis drug can be as described above in the context of the implant. The first phase is combined with a second phase comprising an oil to provide an emulsion, wherein the first phase is a minor phase and the second phase is a major phase.

The solvent used to provide the first phase is capable of dissolving the biodegradable polyester and the disease-modifying osteoarthritis drug. In a specific aspect, the solvent can comprise N-methyl-2-pyrrolidone. Other organic solvents are also contemplated. Preferably the organic solvent is water miscible.

The oil used to form the major phase of the emulsion is a biocompatible oil. Biocompatible oils suitable for use in the present invention include, but are not limited to, medium-chain triglycerides (MCTs) such as caprylic/capric triglyceride, sesame oil, olive oil, castor oil, soybean oil, peanut oil, mineral oil (pharmaceutical grade), safflower oil, sunflower oil, coconut oil, corn oil, jojoba oil, and squalane (hydrogenated squalene). These oils are recognized for their safety, tolerability, and compatibility with biological systems, and are commonly used in pharmaceutical, cosmetic, and biomedical applications. In a specific aspect, the oil can comprise sesame oil.

The method further comprises contacting the emulsion with an aqueous environment to precipitate the plurality of microparticles from the emulsion. In some aspects, the contacting comprises injecting the mixture intraarticularly, and the microparticles are formed in situ in a joint.

The microparticles comprising the biodegradable polyester and the disease-modifying osteoarthritis drug embedded therein can provide a prolonged and sustained release of the drug from the microparticles. In some aspects, the microparticles do not exhibit a burst release profile. For example, in an aspect, less than 25% of the disease-modifying osteoarthritis drug in the microparticles is released from the microparticles after 15 days in an aqueous environment. In some aspects, up to 30 days, or up to 60 days, or up to 90 days are needed for the entirety of the loaded drug to be released.

A plurality of microparticles for intraarticular drug delivery made by the method described herein represents another aspect of the present disclosure. Similar to an intraarticular implant, the intraarticular microparticles can provide therapeutic benefits, including mechanical support, lubrication, drug delivery, or tissue regeneration. The intraarticular microparticles can be solid, semi-solid, or gel-like in nature and may be composed of biocompatible or biodegradable materials. The intraarticular microparticles are capable of release of the DMOAD over a sustained period, effective to modify joint mechanics, or promote cartilage repair and regeneration. The intraarticular microparticles' properties including size, shape, viscoelasticity, and degradation profile can be tailored to ensure compatibility with the joint environment and to minimize adverse tissue reactions.

In an aspect, the intraarticular microparticles can comprise a biodegradable polyester and a disease-modifying osteoarthritis drug contained therein. In some aspects, at least a portion of the biodegradable polyester can be substituted with sucrose acetate isobutyrate. Sucrose acetate isobutyrate is a biodegradable sugar and can be used as a biocompatible matrix or vehicle for controlled release formulations. The biodegradable polyester/sucrose acetate isobutyrate combination, when combined with a DMOAD, can provide a depot system that solidifies or gels upon injection or implantation, creating the desired sustained-release implanted microparticles. Without wishing the be bound by theory, inclusion of the sucrose acetate isobutyrate can facilitate the formation of stable depots that minimize burst release and maintain therapeutic concentrations of the drug at the site of implantation due to its high viscosity and low water solubility.

When present, the sucrose acetate isobutyrate can be included in an amount effective to provide a weight ratio of sucrose acetate isobutyrate:biodegradable polyester of 10:90 to 90:10, or 20:80 to 80:20, or 30:70 to 70:30 or 40:60 to 60:40, or 10:90 to 50:50, or 90:10 to 50:50. Suitable ratios can be selected based on the desired release kinetics, viscosity, and mechanical properties of the depot microparticles.

In an aspect, the disease-modifying osteoarthritis drug comprises a polyphenol, such as punicalagin. Other polyphenols as described herein are also mentioned. In some aspects, the disease-modifying osteoarthritis drug can be present in the intraarticular microparticles in an amount of greater than 0 to 10 weight percent, or greater than 0 to 5 weight percent, or 0.1 to 10 weight percent, or 1 to 10 weight percent, or 0.1 to 5 weight percent, or 1 to 5 weight percent, or 2 to 4 weight percent, each based on the total weight of the intraarticular microparticles.

Another aspect of the present disclosure is a method of treating osteoarthritis by intraarticular injection of punicalagin. In some aspects, the punicalagin can be disposed (e.g., dispersed or encapsulated) in an implant, and the implant may be according to the present disclosure. In some aspects, the punicalagin can be disposed in (e.g., encapsulated in) a plurality of microparticles, and the microparticles can be according to the present disclosure. In some aspects, the implant and the microparticles can comprise poly(lactic-co-glycolic acid), polycaprolactone, or a combination thereof.

This disclosure is further illustrated by the following examples, which are non-limiting.

### EXAMPLES

The present inventor has developed a suitable in situ forming implant for sustained delivery of PCG. Polycarbonate, poly(lactic acid), and poly(lactide-co-glycolide) (PLGA) were dissolved in various solvents and screened for combinations that could also dissolve punicalagin and release it gradually from an implant formed in situ. PLGA emerged as the polymer that offered the best opportunity for the tuning of PCG release kinetics. Systems composed of PLGA and PCG dissolved in N-methyl-2-pyrrolidone (NMP) and/or benzyl alcohol (BA) consistently formed solid or hydrogel implants. 2-Pyrrolidone (2P) also dissolved both PLGA and PCG, but it did not form well-defined implants. Although benzyl benzoate (BB) did not dissolve PCG, its inclusion in the system greatly slowed the rate of PCG release because BB is practically immiscible with water. Thus, one aspect of this invention is the characterization of PLGA in situ forming implants (originating from solutions containing NMP, BA, and BB) with respect to PCG stability and release kinetics.

### Materials and Methods

Two types of LACTEL PLGA were from Evonik Corporation (Birmingham, AL, USA): 50:50 poly(DL-lactide-co-glycolide), inherent viscosity 0.95-1.20 dL/g and 75:25 poly(DL-lactide-co-glycolide), inherent viscosity 0.80-1.20 dL/g, both of which were ester-terminated. Acid-terminated poly(D,L lactide), inherent viscosity 0.16-0.24 dL/g, was from Sigma-Aldrich (St. Louis, MO, USA). NMP, BA, BB, and 2-methyltetrahydrofuran were also from Sigma-Aldrich. PCG (98%) from was from Chengdu Biopurify Phytochemicals Ltd. (Sichuan, China). Collagenase Type 2 was purchased from Worthington Biochemical (Lakewood, NJ, USA), and recombinant human ADAMTS-5 from R&D Systems (Minneapolis, MN). THP-1 human monocytes were from ATCC (Manassas, VA, USA). RPMI 1640 medium, fetal bovine serum (FBS), antibiotic-antimycotic solution, phorbol 12-myristate 13-acetate (PMA), lipopolysaccharides (LPS), dimethyl sulfoxide (DMSO), and Cell Counting Kit-8 (CCK-8) were from Sigma-Aldrich. ELISA kit for Interleukin-1β (IL-1β) was procured from Lifeome Biolabs, Inc. (Oceanside, CA, USA), and a Blyscan™ Glycosaminoglycan Assay kit from Biocolor (Carrickfergus, United Kingdom). LC-MS solvents (water, acetonitrile) and LC-MS grade formic acid were from Fisher Scientific.

### Chondroprotective Properties of PCG

In addition to the inhibition of released collagenases, PCG is capable of suppressing the biosynthesis of inflammatory mediators that play important roles in the pathogenesis of OA. Such chondroprotective properties were investigated using THP-1 monocytes that had been differentiated into macrophages. THP-1 is a human leukemia monocytic cell line. THP-1 monocytes were expanded by suspension culture in RPMI 1640 medium supplemented with 10% FBS and 1% antibiotic-antimycotic solution. They were differentiated into macrophages by adding PMA to a final concentration of 20 nM and transferring them to monolayer culture for 72 h.¹³ Inflammation was then induced by treatment with 1 μg/ml LPS. PCG was first dissolved at 5 mM in complete culture medium. One hour after LPS stimulation, cells were treated with PCG at final concentrations of 4, 20, 100, and 500 μM. Culture supernatant was collected 24 h later and IL-1β quantified using ELISA, with results expressed as a percentage of the unstimulated control.

In addition to IL-1β, prostaglandin E2 (PGE2) in the culture supernatants was quantified using mass spectrometry. The same culture supernatant used for the IL-1β ELISA was fortified with a cocktail of deuterated internal standards (TxB2-d4, PGE2-d4, 15-HETE-d8, AA-d8, 100 pmol each, Cayman Chemicals). An equal volume of 3:2 hexane: isopropanol (v/v)+0.1% acetic acid was mixed with the medium by vortex mixing for 1 min. Following centrifugation of the mixture (800×g, 5 min), the top organic layer was collected and the medium extracted again with an equal volume of hexane. The subsequent hexane layer was combined with the first extract and the organics evaporated to dryness under nitrogen. The residues were then reconstituted in 100 μL of methanol and analyzed by liquid chromatography with tandem mass spectrometry (LC-MS/MS), as previously described.¹³

### Implant Formation and Measurement of Released PCG

Implant formulations are specified by the volume ratio of solvents and mass concentration of polymer and PCG. Total solvent for each batch was 350-500 μl. After solvents were mixed in the desired v/v ratio in a microcentrifuge tube, 12 mg of PCG was added. The appropriate amount of polymer was calculated based on the desired mass concentration (15-25% w/w) and the solvents' densities. The polymer was allowed to dissolve overnight in a 70° C. water bath. The solvent/drug/polymer mixture was stirred to achieve a homogeneous solution. A single implant was produced by injecting or pipetting one-third of the solution into a 13×100 mm disposable glass culture tube containing 3 ml distilled water with 0.25% sodium dodecyl sulfate (SDS). Thus, three implants were obtained from each batch. The tubes were kept at 37° C. under orbital shaking at 100 rpm in a Thermo Scientific MaxQ 4000 Incubated/Refrigerated Shaker (Marietta, OH, USA). Release of PCG was characterized by periodically measuring the absorbance of the bathing solution at 378 nm using a Bio-Tek μQuant Universal Microplate Spectrophotometer (Winooski, VT, USA). PCG concentration was determined by comparison to a standard curve produced from serial dilutions of a PCG/distilled water solution.

### Effects of Implant Formulation on Kinetics of PCG Release

Several experiments were performed to gain a better understanding of how implant formulation affects the release of PCG (Table 1). The long-term goal is to find a formulation that sustains steady release of PCG for up to 100 days with minimal burst release. The first experiment was to determine if either NMP or BA solvents were suitable by themselves when holding the polymer type and its concentration constant. PLGA 75:25 was the default polymer type because degradation by hydrolysis generally takes longer the higher the lactide: glycolide ratio. Note that BB could not be tested as the only solvent because it did not dissolve PCG. The next experiment was to determine the general effect of solvent water miscibility by changing the NMP/BB ratio while holding polymer type and concentration constant. Whereas BB is practically immiscible with water, NMP is completely miscible. Because it does not dissolve PCG, BB did not make up more than 50% of the total solvent. The effect of polymer type was then examined while holding BB constant at the intermediate level of 30%. PLA was included because it had been shown to be advantageous compared to PLGA for sustained delivery of a relatively high molecular weight drug using in situ forming microparticle systems.¹⁴ Having demonstrated a slight advantage of PLGA 75:25 over the others, an additional experiment was carried out to determine the effect of polymer concentration, again holding NMP/BB constant at 70/30. A final experiment was performed as a first step towards optimization. BA, which has moderate solubility in water, was included to replace a majority of the highly water soluble NMP.

TABLE 1

| | Polymer | Solvents |
|---|---|---|
| Experiment | (Concentration) | (Volume Ratio) |
| Effect of single | PLGA 75:25 (20%) | NMP |
| solvent | PLGA 75:25 (20%) | BA |
| Effect of solvent | PLGA 75:25 (20%) | NMP/BB (50/50) |
| water miscibility | PLGA 75:25 (20%) | NMP/BB (70/30) |
| | PLGA 75:25 (20%) | NMP/BB (90/10) |
| Effect of polymer | PLGA 50:50 (20%) | NMP/BB (70/30) |
| type | PLGA 75:25 (20%) | NMP/BB (70/30) |
| | PLA (20%) | NMP/BB (70/30) |
| Effect of PLGA | PLGA 75:25 (15%) | NMP/BB (70/30) |
| concentration | PLGA 75:25 (20%) | NMP/BB (70/30) |
| | PLGA 75:25 (25%) | NMP/BB (70/30) |
| Optimization | PLGA 75:25 (20%) | NMP/BA/BB |
| of PCG release | | (20/40/40) |

PCG Stability within In Situ Forming Implants (ISIs)

The stability of PCG within a PLGA implant was examined by determining the ability of released PCG to inhibit collagenase-mediated cartilage degradation. Pig stifle (knee) joints were obtained from a local meat processor and full-thickness, cylindrical disks of articular cartilage (5 mm diameter) were harvested from the distal femurs. The disks were freeze-dried, weighed, and placed in Dulbecco's Modified Eagle Medium (DMEM) containing 0.0025% type 2 collagenase with and without 100 μM PCG (n=12 disks per group). Samples were incubated at 37° C. under gentle orbital shaking. At 3, 6, 9, and 12 days of incubation, three disks from each group were rinsed in distilled water, freeze-dried and weighed again, while the rest were allowed to continue incubation after adding fresh collagenase. The same experiment was repeated using implants made with 20% PLGA (50:50), NMP/BB 90/10, and 0 or 32 mg PCG per gram total implant weight (n=12 per group). Implants were formed in microcentrifuge tubes by pipetting 100 μl of the solution into distilled water. One hour later the water was replaced by the collagenase solution. Custom 3D-printed baskets were used to suspend the cartilage disks in the collagenase just above the implants. Disks were collected and weighed at the same 3-day intervals, with replacement of the collagenase solution each time. PCG concentration in the spent collagenase solution was determined by measuring absorbance at 373 nm and comparing to a standard curve generated from serial dilutions of PCG dissolved directly into DMEM. For both experiments, data are reported as residual mass of cartilage (final weight/initial weight). The overall effect of PCG was analyzed by two-way ANOVA with treatment and time as factors. Statistically significant effects of PCG at each time point were determined by independent t-test (equal variances not assumed).

### LC-MS/MS Analysis of Punicalagin

PCG levels in water were determined by injecting 10 μL of the samples onto a Waters BEH C18 column (2.1×50 mm) and chromatography using a Waters UPLC that was interfaced with a Thermo Quantis triple-quadrupole mass spectrometer. The LC mobile phase solvents were 95:5 H2O/ACN+0.1% formic acid (mobile phase A) and 95:5 ACN/H2O+0.1% formic acid (mobile phase B). The gradient program used was as follows: 0-0.5 min (100% A, 0% B), 0.5-4.9 min (70% A, 30% B), 4.9-5.0 (70% A, 30% B), 5.0-5.1 min (100% A, 0% B), with column equilibration for 5 min. The column temperature was held at 30° C. and the mobile phase flow rate was 0.2 mL/min. PCG was analyzed by negative electrospray ionization using the precursor-product transition 1083.1>600.8. An external calibration curve was prepared for PCG in water to enable its concentration in the experimental samples to be estimated.

### Injectability

From Table 1, implants formulated to optimize PGC release were evaluated for their injectability. Injection forces were measured for implants formed using NMP/BB/BA (20/40/40) at 20% w/w PGLA (75:25). The solution was prepared as above; however, PCG was not added for the purpose of determining injectability. Hydrogel implants were formed by injecting 750 μl of the formulation into 10 ml of distilled water, followed by incubation at 37° C. under gentle orbital shaking for 24 hours (n=3). Each implant was pipetted into a 5 ml syringe following removal of the plunger. All air was aspirated from the syringe following the replacement of the plunger. The syringe was mounted upright in a Mach-1 V500C Mechanical Tester (Biomomentum, Inc., Laval, QC, Canada). A flat, smooth plate was lowered onto the plunger with a contact force of 0.098 N. The force to expel the implant through a 21G×1.5 inch needle was then measured at a frequency of 100 Hz as the plunger was depressed at rates of 0.1, 0.3, and 0.5 mm/s Thirty seconds was the maximum time allowed for injection. Thus, the plunger was advanced a distance of 3 mm for each trial, which resulted in a total expelled implant volume of 400 μl. Friction of the plunger moving in an empty syringe, as well as the resistance of water, were tested for comparison.

### Cytotoxicity

Cytotoxicity was examined using fresh porcine articular cartilage explants. As a promising candidate with respect to PCG release kinetics, implants composed of PLGA (75:25) dissolved in NMP/BA/BB (20/40/40) at 20% w/w were prepared as above in a 24-well tissue culture plate. The cytotoxicity experiment was carried out using ISIs without PCG in order to isolate the effects of the drug delivery system. An implant of 70 μl was formed in each well. They were prepared 2, 24, 48, and 72 hours ahead of exposure to cartilage. The water/SDS was discarded and replaced with PBS every 24 hours and/or 1 hour prior to the introduction of explants. At the time of explant harvest, PBS was replaced with 1 ml of DMEM containing 10% FBS. Full-thickness disks of cartilage, 4 mm in diameter, were harvested from the medial condyle of the distal femur using a biopsy punch and scalpel. They were placed directly into the wells containing implants. Control disks were cultured in wells without implants. Explants were cultured for 24 hours in the presence of implants, at which time they were transferred to a new plate for viability assessment using the CCK-8 assay. Absorbance (450 nm-650 reference) for each experimental sample was normalized to the average control absorbance, and results expressed as percent viability relative to control. Statistically significant differences with respect to control viability were determined using one-way ANOVA and Dunnett posthoc t-tests.

### Results

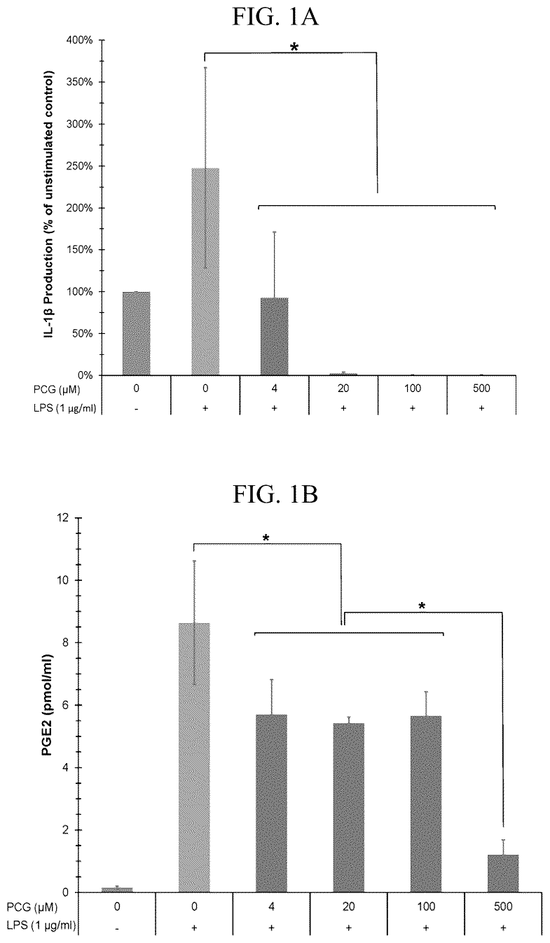

Chondroprotective Properties of PCG. The anti-inflammatory nature of PCG was demonstrated by stimulating THP-1 macrophages with LPS. As shown in FIG. 1A, PCG reduced LPS-stimulated IL-1β production by THP-1 macrophages in a concentration-dependent manner, with return to the unstimulated control level at 4 μM and suppression of practically all IL-1β synthesis at 20 μM and above. PCG's effect on PGE2 synthesis was not as pronounced (FIG. 1B), but it did suppress PGE2 levels. In the range of 4-100 μM, PCG decreased PGE2 production by about 35%. However, at 500 μM PCG, the LPS-stimulated production of PGE2 was attenuated by approximately 85%.

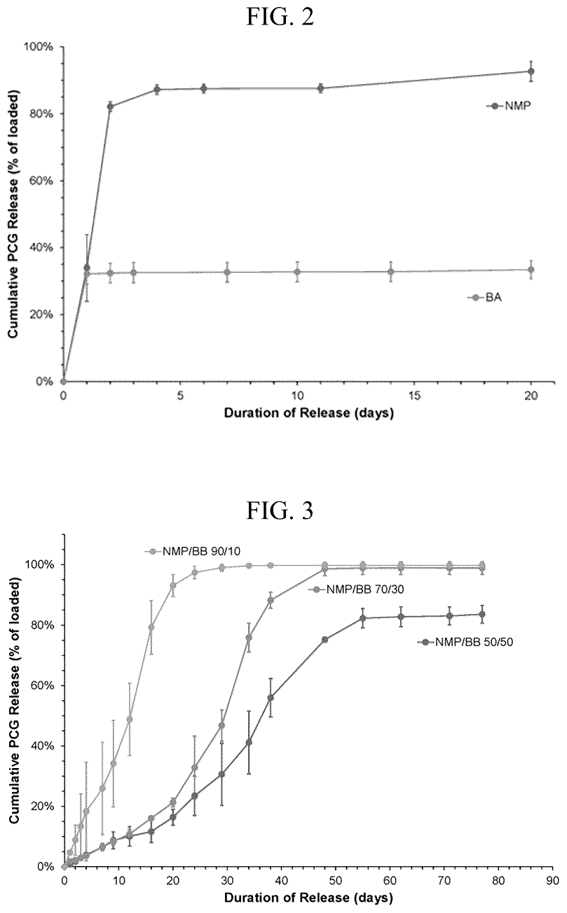

NMP or BA as lone solvent. When PCG and PLGA were dissolved in NMP solvent alone, phase inversion occurred very rapidly but the implants were immediately hard and crumbly. Those formed from PCG and PLGA dissolved in BA alone were initially soft hydrogels that contracted and stiffened over the course of several days. PCG release profiles are shown in FIG. 2. Both exhibited a burst biphasic drug release profile.¹⁵ In the case of NMP, a 2-day burst accounted for over 80% of the PCG loaded. The small amount remaining released quite slowly, and 95% had released within 20 days. For BA, approximately one-third of the PCG loaded was released in burst fashion. Thereafter the rate of release was practically zero. Furthermore, the remaining PCG was not recovered in the 20-day implants upon dissolving them in NMP.

Effect of solvent miscibility with water. BB is practically immiscible in water, and it was used to alter the overall solvent water miscibility by mixing it with NMP in various proportions. Results are summarized in FIG. 3. With the addition of BB to NMP, PCG displayed sigmoidal shaped release curves that indicate a delayed biphasic release profile characterized by an initial lag period followed by a rapid release in the power-law phase.¹⁵ The power-law phase refers to a period during which (Δc)˜(Δt)ⁿ, where Δc is the cumulative amount of drug released and Δt is the elapsed time.¹⁵ There was a clear trend in increasing lag period with increasing BB content (lower water miscibility). Sustained PCG release for approximately 55 days was achieved using NMP: BB 50:50.

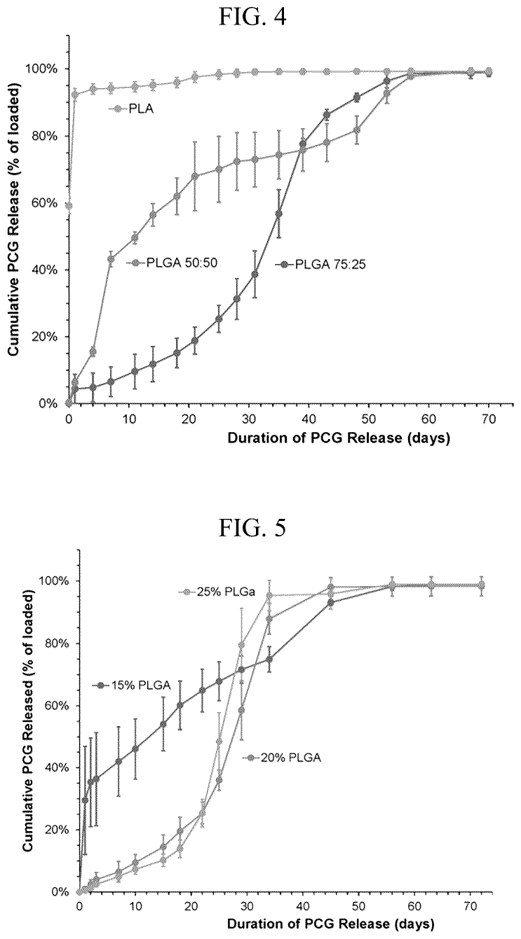

Effect of polymer type. The effect of polymer type on PCG release kinetics is shown in FIG. 4. PLA implants were not well-defined and turned the water cloudy. The profile was burst biphasic, with approximately 60% of the loaded PCG detected in the water just two hours after implant formation, and the majority of what remained released within 24 hours. PLGA 50:50 and PLGA 75:25 were able to sustain PCG release for almost 60 days, but the release profiles were different. Whereas PLGA 75:25 produced a classic delayed biphasic profile, PCG release from PLGA 50:50 was roughly triphasic with burst, power-law, and accelerated release phases.¹⁵

Effect of PLGA concentration. The influence of PLGA concentration on PCG release kinetics is shown in FIG. 5. Implants created from 15% PLGA displayed a biphasic, perhaps triphasic, drug release profile, which included burst release of approximately one-third of the loaded PCG in two days. In contrast, PCG released from 20% and 25% PLGA implants in a delayed biphasic manner, and there was very little difference between the two higher concentrations.

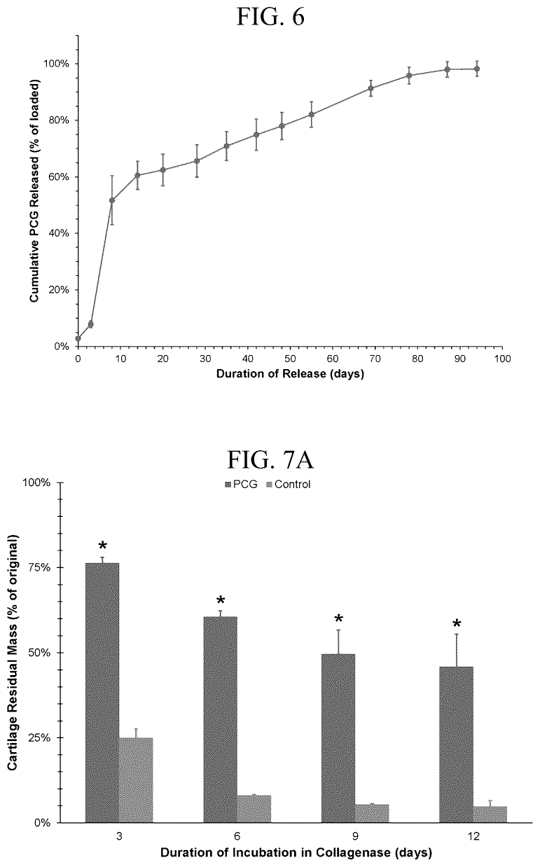

Optimization of PCG release profile. The ideal PCG release profile would be monophasic, requiring several months to deliver the supply of drug. Based on the previous results, the first attempt to optimize the PCG release involved blending NMP, BA, and BB in a 20:40:40 ratio. Since little difference was observed between 20% and 25% PLGA, 20% was selected to facilitate dissolution and pipetting. The shape of the release profile (FIG. 6) does not closely resemble any of the typical shapes of in vitro cumulative release profiles that represent known types of drug release behavior from PLGA delivery systems. Release followed a pattern of short lag, burst, and then steady release between 14 and 80 days. Overall, PCG release was sustained for approximately 90 days in vitro.

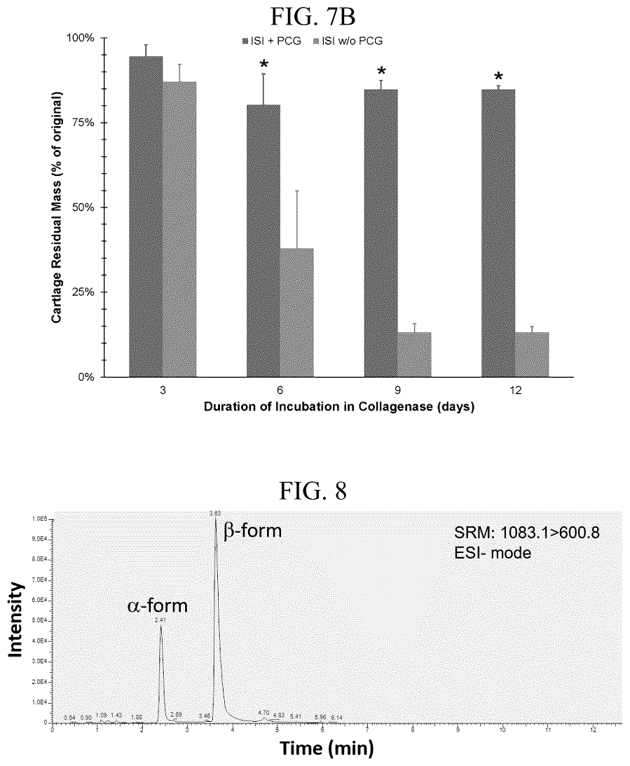

PCG Stability within ISI's. PCG significantly inhibited collagenase-mediated degradation of cartilage, whether it was added directly to the collagenase solution (FIG. 7A) or released from an ISI (FIG. 7B). The addition of 100 μM PCG limited cartilage mass loss to about 55% over 12 days of exposure to 0.0025% type 2 collagenase, whereas control cartilage disks lost approximately 95% of their mass in the same span of time. In the presence of an ISI containing PCG, the average cartilage disk exposed to collagenase for 12 days retained over 85% of its original mass. Control disks incubated in collagenase with ISI's without PCG retained less than 10% of their original mass. The concentration of PCG released into the collagenase solution, as determined by UV/Vis spectrometry, was 158, 152, 82, and 96 μM on Days 3, 6, 9, and 12, respectively. These results demonstrate that PCG is stable within the PLGA ISI's for at least 12 days.

LC-MS/MS analysis confirmed the presence of punicalagin in the release medium (water) from an implant incubated under sink conditions for 21 days (FIG. 8). Punicalagin undergoes tautomerism; therefore, it exists as both α- and β-anomers in aqueous solution, which are detected as unique LC-MS/MS peaks. Total PCG concentration on Day 21, estimated by summing a and β peaks, was 195 μM, compared to 155 μM on Day 7. In both instances, the water had been completely removed and replaced with fresh water 4 days prior to collection. Thus, these results not only demonstrate the stability of PCG within the ISI, but also show that PCG release was somewhat steady during the 21-day evaluation period.

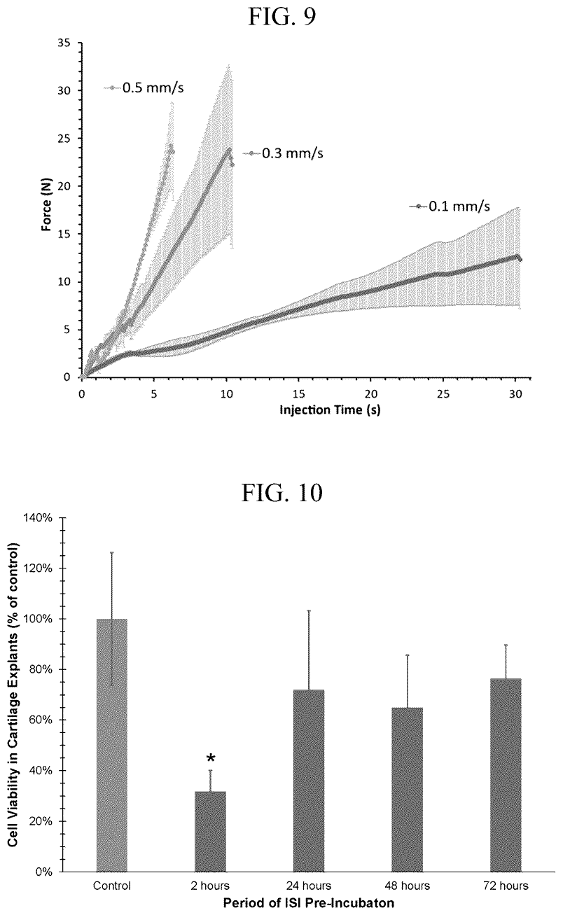

Injectability. The ideal ISI formulation would require similar injection forces to water or other commonly intraarticularly injected solutions, such as hyaluronic acid or corticosteroids. Additionally, the injection rate should allow an ISI to be fully injected within a reasonable time. Injection forces were found to be directly proportional to the injection rate for ISI, with a peak of 12.7 N at 0.1 mm/s and a peak force of 24.2 N reached during injection at 0.5 mm/s (FIG. 9). For comparison, a peak injection force for water at 0.5 mm/s injection rate was 1.23 N. These results demonstrate the ability to inject ISI through needles used during intraarticular injections.

Cytotoxicity. Preliminary experiments demonstrated that implants were highly cytotoxic unless they were allowed to undergo a period of phase inversion to eliminate a substantial amount of solvent. Therefore, they were tested for cytotoxicity up to 72 hours after formation. As shown in FIG. 10, a period maturation of 24 hours or more drastically decreases the implant's cytotoxicity to primary chondrocytes. The data suggest that a period of pre-incubation ≥24 hour to allow for removal of solvent did not render the implants completely safe, but significantly decreased viability could not be demonstrated given the degree of variability.

### DISCUSSION

Punicalagin is the major polyphenol present in pomegranate (Punica granatum L.) and contributes to its anti-inflammatory properties.⁸ PCG is of interest as a DMOAD because there are multiple mechanisms by which it may be able to protect cartilage from degradation. Importantly, PCG has been shown to inhibit the production of pro-inflammatory cytokines, including TNF-α, IL-6, and IL-1β, by attenuating NF-κB/iNOS/COX-2/TNF-α and mitogen-activated protein kinases (MAPK) signaling pathways.^{16,17} Suppression of NF-κB/iNOS/COX-2/TNF-α also lowers the production of PGE2, which, if left unchecked, enhances the expression of MMP-13 and ADAMTS-5 and accelerates extracellular matrix (ECM) degradation.¹⁸ Furthermore, PCG can directly inhibit the secreted enzymes that degrade the cartilage ECM in OA. PCG forms complexes with collagenases in solution, including MMP-13, and inhibits their enzymatic activity.^{11,19} Another way PCG can protect cartilage is by acting as a scavenger of reactive oxygen species. Punicalagin possesses multiple phenol groups that can participate in redox reactions by donating hydrogen atoms to oxidizing agents. Thus, it has potent electron-donating capacity, enabling it to efficiently reduce and detoxify free radicals, sparing vital macromolecules from attack. Finally, PCG binding to cartilage collagen may limit access to degradative enzymes. PCG binds to collagen type II with high affinity, which occurs through formation of multiple hydrogen bonds (PCG has 17 hydroxyl groups), in addition to x-x and electrostatic interactions.¹¹

There is evidence that PCG is effective for the treatment of OA. Orally administered pomegranate fruit extract has been shown to lessen the severity of induced OA in rats and rabbits.^{9,10} Daily intraperitoneal doses of PCG at 50 mg/kg significantly reduced paw edema in an adjuvant-induced arthritis rat model.¹¹ Our own study showed that semi-weekly injections of a solution containing 9.2 mM PCG seemed to result in less overall erosion of cartilage compared to a saline control in a rat model of osteoarthritis.¹⁹ As part of an investigation of PCG as a potential therapeutic compound for relieving rheumatoid arthritis, PCG was found to reduce the TNF-α induced expression of IL-1β, IL-6, and MMP-13 in fibroblast synoviocytes.²⁰ Such studies point to PCG as a promising DMOAD. Moreover, PCG safety is well established. For example, cytotoxicity is very low,²¹ and hematological and histopathological analysis of rats fed a 6% w/w PCG-containing diet for 37 days indicated no systemic toxicity.²² Likewise, histology of major organs in our study turned up no evidence of systemic toxicity.¹⁹

The present invention also demonstrates additional chondroprotective properties of PCG. In a previous study, it was reported that PCG attenuated the LPS-induced production of IL-1β and PGE2 in RAW264.7 murine macrophages through disruption of NF-κB signaling pathway.¹⁷ IL-1β is among the main proinflammatory cytokines implicated in OA.¹⁷ It triggers the secretion of additional inflammatory mediators and enzymes that degrade cartilage.¹⁶ Specifically, IL-1β activates chondrocytes, the cells responsible for maintaining cartilage, to increase the production of matrix metalloproteinases (MMPs), particularly MMP-1, MMP-3, and MMP-13. These enzymes target and degrade key components of the cartilage ECM, such as collagen fibers and proteoglycans. Furthermore, IL-1β stimulates the synthesis of prostaglandins and nitric oxide, which contribute to cartilage breakdown and inhibit the production of collagen and proteoglycans, essential for maintaining cartilage integrity. Eicosanoids, such as prostaglandins and leukotrienes, are lipid-based signaling molecules and mediators of inflammation. Substantially increased synthesis of eicosanoids is associated with progression from acute to chronic inflammation.⁵² Results presented herein show that PCG has suppressive effects on the LPS-induced production of IL-1β and PGE2 in human THP-1 macrophages. Thus, PCG's persistence within a joint could reasonably be expected to decrease cartilage degeneration.

While oral administration of PCG is possible, it relies on transport through the systemic circulation to reach the intraarticular site of action. Localized delivery via intraarticular injection maximizes drug activity at the target location while minimizing exposure of other organs and the risk of unwanted side effects.⁴ Furthermore, orthopedists are trained to perform intraarticular injections for viscosupplementation, and the procedure is safe with low risk of infection.^{5,6} Unfortunately, intraarticularly injected small molecule drugs are rapidly cleared from the synovial space via efficient lymphatic drainage. Therefore, it is highly advantageous that the drug be immobilized in an injectable depot formulation from which it undergoes sustained release. The present invention shows the potential for PLGA-based ISI's to act as such a depot. PLGA is an FDA-approved biopolymer, and PLGA ISI's can deliver hydrophilic or hydrophobic drugs. They generally sustain release for much longer than conventional drug delivery systems. In situ forming implants have been administered by oral, ocular, rectal, vaginal, injection (intramuscular, subcutaneous) and intraperitoneal routes.^{2,3}

NMP and BA are solvents for both PLGA and PCG, but neither produced suitable implants by themselves as most PCG was released in burst fashion. Being highly miscible with water, NMP forms a drug depot very quickly, but it is highly porous and permeable. A high burst release is a consistent feature of ISI's containing NMP and has been observed for polycaprolactone, polylactide, and poly(trimethylene carbonate), in addition to PLGA.²⁴ With BA (very low water miscibility), because the rate of solvent exchange was much slower, a high proportion of PCG may have transferred into the aqueous medium before it became encapsulated. A mixture of NMP and BB, however, produced implants that could sustain PCG delivery for many weeks. It seems that PCG was encapsulated quickly but that the BB, which is practically immiscible with water, promoted the formation of a dense implant from which PCG diffused slowly. ISIs containing BB alone could not be investigated because BB did not dissolve PCG.

Because almost all loaded PCG released in burst fashion, a particular PLA used in connection with this invention did not form a suitable drug depot. A difference in solubility may have been the culprit, but it was not tested. ISIs made from PLGA 50:50 and PLGA 75:25 both sustained release of PCG for several weeks, but the patterns of release were different. As over half of the loaded PCG released from the PLGA 50:50 within two weeks, PLGA 75:25, from which approximately 12% of the loaded PCG released in that period, is the preferred candidate for further development.

With respect to PLGA concentration, burst release of PCG was mitigated by an increase from 15% to 20%, but a further increase to 25% had no additional effect. The higher concentrations of PLGA (20%, 25%) formed highly viscous solutions, and it was difficult to work with concentrations above 25%. Based on results of system characterization studies, one attempt was made at optimization. ISIs composed of PLGA (75:25) dissolved in NMP/BA/BB 20/40/40 at 20% w/w released PCG for approximately 90 days. However, about 50% of it released in under 10 days.

Stability of the PCG within the ISI is an important feature, as interaction with excipients can lead to drug degradation or inactivation.²⁵ Judging from the collagenase inhibition experiment, there was no loss of functionality of PCG released from PLGA ISIs. ISIs were highly chondroprotective of cartilage explants exposed to type 2 collagenase for 12 days. In fact, there was no evidence of collagenase-induced erosion from Day 6 to Day 12. Day 12 explants retained approximately 85% of their original mass compared to about 15% for controls. LC-MS/MS confirmed the release of undegraded PCG during Day 3-7 and Day 17-21 intervals. Furthermore, the concentration of PCG detected was consistent with that measured after 3-day periods of release as part of the collagenase inhibition experiment. PCG's many hydroxyl groups could interact catalytically to increase exposure of PLGA's ester linkages to water, thereby facilitating PLGA hydrolysis.²⁵

The aforementioned ISI capable of sustaining PCG release in vitro for up to 90 days was further investigated with respect to injectability. For all rates tested, the force required to depress the plunger steadily increased, and the faster the ejection rate, the higher was the rate of force increase. During the injection of 400 μl through a 21G needle, the average peak forces ranged from approximately 13 to 24 N. For reference, data from ten evaluators were used to establish the upper threshold force for smooth subcutaneous injection as 35 N, with injection feasible up to 45 N, albeit with difficulty.²⁷ Extrapolating the data from the 0.5 mm/s tests, the force required to inject a 1 ml ISI solution in about 10 s would just reach 45 N, assuming the joint space did not offer substantial resistance.

When considering the intraarticular route of administration, cytotoxicity of the excipients is perhaps the issue of greatest concern for an ISI. Necrotically dying chondrocytes spill their contents upon lysis, and the intracellular components activate inflammatory signaling pathways. Furthermore, a loss of chondrocytes would result in reduced cartilage matrix turnover and capacity for maintenance and repair. Thus, excipient cytotoxicity could easily overwhelm whatever chondroprotective effects PCG may have. Unfortunately, the organic solvents needed to dissolve PLGA are highly cytotoxic. A degree of chondrocyte survival was achieved only when some time was allowed for solvent exchange with water to occur prior to cartilage explant exposure. However, a waiting period of 72 hours was insufficient to prevent a 20% decrease in viability of ISI-exposed chondrocytes relative to controls.

These data suggest that alternative approaches, such as in situ forming microparticles, may be needed. In such systems, dissolved PLGA constitutes an internal phase, which is emulsified into a biocompatible external oily phase such as sesame oil. When the emulsion is injected into an aqueous environment, solvent diffuses out of the droplets, and the PLGA and drug precipitate and form microparticles. In situ forming microparticles are reported to have lower toxicity compared to ISIs.²⁸ Furthermore, they may be made even less cytotoxic by incorporating a component such as sucrose acetate isobutyrate, which dissolves in a much smaller amount of organic solvent than PLGA, to replace a substantial amount of the PLGA.²⁸

In conclusion, the present invention demonstrates that PCG suppressed production of IL-1β and PGE2 by human THP-1 macrophages and that PCG was stably incorporated into PLGA-based ISIs for controlled release. PCG release from ISIs is tunable, and at least one formulation sustained release for up to 90 days. The force needed to inject ISI's of the same formulation is marginally acceptable, and cytotoxicity was greatly reduced when the ISI was allowed at least 24 h for removal of solvent before its presentation to cartilage explants. Overall, this invention shows that PCG-releasing ISIs are a useful, novel approach to localized, long-lasting release of a DMOAD.

### REFERENCES

1. Safiri S, Kolahi A-A, Smith E, et al. Global, regional and national burden of osteoarthritis 1990-2017: a systematic analysis of the Global Burden of Discase Study 2017. Ann Rheum Dis. 2020; 79 (6): 819-828.
2. Collaborators G 2015 D and II and P. Global, regional, and national incidence, prevalence, and years lived with disability for 310 diseases and injuries, 1990-2015: a systematic analysis for the Global Burden of Discase Study 2015. Lancet. 2016; 388 (10053): 1545-1602.
3. Hunter D, Schofield D, Callander E. The individual and socioeconomic impact of osteoarthritis. Nat Rev Rheumatol. 2014; 10:437-441.
4. Evans C H, Kraus V B, Setton L A, Rheumatol N R. Progress in intraarticular therapy. Nat Rev Rheumatol. 2014; 10(1):11-22. doi: 10.1038/nrrheum.2013.159
5. Pal B, Morris J. Perceived risks of joint infection following intraarticular corticosteroid injections: A survey of rheumatologists. Clin Rheumatol. 1999; 18(3):264-265. doi: 10.1007/s100670050098
6. Amin N H, Omiyi D, Kuczynski B, Cushner F D, Scuderi G R. The Risk of a Deep Infection Associated With Intraarticular Injections Before a Total Knee Arthroplasty. J Arthroplasty. 2016; 31(1):240-244. doi: 10.1016/j.arth.2015.08.001
7. McHugh A J. The role of polymer membrane formation in sustained release drug delivery systems. J Control Release. 2005; 109(1-3):211-221. doi: 10.1016/j.jconrel.2005.09.038
8. BenSaad L A, Kim K H, Quah C C, Kim W R, Shahimi M. Anti-inflammatory potential of ellagic acid, gallic acid and punicalagin A&amp; B isolated from Punica granatum. BMC Complement Altern Med. 2017; 17(1):47. doi:10.1186/s12906-017-1555-0
9. Akhtar N, Khan N M, Ashruf O S, Haqqi T M. Inhibition of cartilage degradation and suppression of PGE2 and MMPs expression by pomegranate fruit extract in a model of posttraumatic osteoarthritis. Nutrition. 2017; 33:1-13. doi: 10.1016/j.nut.2016.08.004
10. Lec C J, Chen L G, Liang W L, Hsich M S, Wang C C. Inhibitory effects of punicalagin from Punica granatum against type II collagenase-induced osteoarthritis. J Funct Foods. 2018; 41:216-222. doi: 10.1016/j.jff.2017.12.026
11. Jean-Gilles D, Li L, Vaidyanathan V G G, et al. Inhibitory effects of polyphenol punicalagin on type-II collagen degradation in vitro and inflammation in vivo. Chem Biol Interact. 2013; 205 (2): 90-99. doi: 10.1016/j.cbi.2013.06.018
12. Elder S H, Mosher M L, Jarquin P, Smith P, Chironis A. Effects of short-duration treatment of cartilage with punicalagin and genipin and the implications for treatment of osteoarthritis. J Biomed Mater Res—Part B Appl Biomater. 2021; 109 (6): 818-828. doi: 10.1002/jbm.b.34747
13. Wang R, Borazjani A, Matthews A T, Mangum L C, Edelmann M J, Ross M K. Identification of palmitoyl protein thioesterase 1 in human thp1 monocytes and macrophages and characterization of unique biochemical activities for this enzyme. Biochemistry. 2013; 52 (43): 7559-7574. doi: 10.1021/bi401138s
14. Luan X, Bodmeier R. In situ forming microparticle system for controlled delivery of leuprolide acetate: Influence of the formulation and processing parameters. Eur J Pharm Sci. 2006; 27 (2-3): 143-149. doi: 10.1016/j.ejps.2005.09.002
15. Yoo J, Won Y Y. Phenomenology of the Initial Burst Release of Drugs from PLGA Microparticles. ACS Biomater Sci Eng. 2020; 6 (11): 6053-6062. doi: 10.1021/acsbiomaterials.0c01228
16. Xu J, Cao K, Liu X, Zhao L, Feng Z, Liu J. Punicalagin regulates signaling pathways in inflammation-associated chronic diseases. Antioxidants. 2022; 11 (1): 1-12. doi: 10.3390/antiox 11010029
17. Xu X, Yin P, Wan C, et al. Punicalagin inhibits inflammation in LPS-induced RAW264.7 macrophages via the suppression of TLR4-mediated MAPKs and NF-κB activation. Inflammation. 2014; 37 (3): 956-965. doi: 10.1007/s10753-014-9816-2
18. Attur M, Al-mussawir H E, Patel J, et al. Cartilage: Evidence for Signaling via the EP4 Receptor 1. J Immunol. 2008; 181:5082-5088. http://www.jimmunol.org/content/181/7/5082.
19. Elder S, Mosher M, Jarquin P, Smith P, Chironis A. Effects of short-duration treatment of cartilage with punicalagin and genipin and the implications for treatment of osteoarthritis. J Biomed Mater Res—Part B Appl Biomater. 2021; 109 (6): 818-828.
20. Huang M, Wu K, Zeng S, et al. Punicalagin inhibited inflammation and migration of fibroblast-like synoviocytes through nf-kb pathway in the experimental study of rheumatoid arthritis. J Inflamm Res. 2021; 14:1901-1913. doi: 10.2147/JIR.S302929
21. Kulkarni A P, Mahal H S, Kapoor S, Aradhya S M. In vitro studies on the binding, antioxidant, and cytotoxic action of punicalagin. J Agric Food Chem. 2007; 55 (4): 1491-1500. doi: 10.1021/jf0626720
22. Cerdá B, Cerón JJ, Tomás-Barberán FA, Espín JC. Repeated oral administration of high doses of the pomegranate ellagitannin punicalagin to rats for 37 days is not toxic. J Agric Food Chem. 2003; 51 (11): 3493-3501. doi: 10.1021/jf020842c
23. Madan M, Bajaj A, Lewis S, Udupa N, Baig JA. In situ forming polymeric drug delivery systems. Indian J Pharm Sci. 2009; 71 (3): 242-251. doi: 10.4103/0250-474X.56015
24. Zhang X, Yang L, Zhang C, et al. Effect of polymer permeability and solvent removal rate on in situ forming implants: Drug burst release and microstructure. Pharmaceutics. 2019; 11 (10). doi: 10.3390/pharmaceutics11100520
25. Parent M, Nouvel C, Koerber M, Sapin A, Maincent P, Boudier A. PLGA in situ implants formed by phase inversion: Critical physicochemical parameters to modulate drug release. J Control Release. 2013; 172 (1): 292-304. doi: 10.1016/j.jconrel.2013.08.024
26. Ahmed T A, Ibrahim H M, Samy A M, Kascem A, Nutan M T H, Hussain M D. Biodegradable injectable in situ implants and microparticles for sustained release of montelukast: In vitro release, pharmacokinetics, and stability. AAPS PharmSciTech. 2014; 15 (3): 772-780. doi: 10.1208/s12249-014-0101-3
27. Cilurzo F, Selmin F, Minghetti P, et al. Injectability evaluation: An open issue. AAPS PharmSciTech. 2011; 12 (2): 604-609. doi: 10.1208/s12249-011-9625-y
28. Haider M, Elsayed I, Ahmed I S, Fares A R. In situ-forming microparticles for controlled release of rivastigmine: In vitro optimization and in vivo evaluation. Pharmaceuticals. 2021; 14 (1): 1-21. doi: 10.3390/ph14010066

This disclosure further encompasses the following aspects.
Aspect 1: A method of forming an implant, the method comprising: providing a mixture comprising a water-miscible organic solvent; a water-immiscible organic solvent; a biodegradable polyester comprising poly(lactic-co-glycolic acid), polycaprolactone, or a combination thereof; and a disease-modifying osteoarthritis drug; and contacting the mixture with an aqueous environment to form a precipitate from the mixture to provide the implant.
Aspect 2: The method of aspect 1, wherein the water-miscible organic solvent comprises N-methyl-2-pyrrolidone.
Aspect 3: The method of aspect 1 or 2, wherein the water-immiscible organic solvent comprises benzyl benzoate, benzyl alcohol, or a combination thereof.
Aspect 4: The method of any of aspects 1 to 3, wherein the biodegradable polyester comprises poly(lactic-co-glycolic acid) having a lactide: glycolide ratio of 25:75 to 75:25.
Aspect 5: The method of any of aspects 1 to 4, wherein the mixture comprises N-methyl-2-pyrrolidone, benzyl benzoate, and benzyl alcohol.
Aspect 6: The method of aspect 5, wherein a weight ratio of N-methyl-2-pyrrolidone:benzyl benzoate:benzyl alcohol is 10-30:30-50:30-50, preferably 15-25:35-45:35:35, more preferably 20:40:40.
Aspect 7: The method of any of aspects 4 to 6, wherein the poly(lactic-co-glycolic acid) is present in an amount of 15 to 30 weight percent, based on the total weight of the mixture.
Aspect 8: The method of any of aspects 4 to 7, wherein the poly(lactic-co-glycolic acid) is present in an amount of 20 to 25 weight percent, based on the total weight of the mixture.
Aspect 9: The method of any of aspects 1 to 8, wherein the disease-modifying osteoarthritis drug is punicalagin.
Aspect 10: The method of any of aspects 1 to 9, wherein the mixture is homogenous.
Aspect 11: The method of any of aspects 1 to 10, wherein the mixture is injectable.
Aspect 12: The method of any of aspects 1 to 11, wherein the contacting comprises injecting the mixture intraarticularly, and the implant is formed in situ in a joint.
Aspect 13: The method of any of aspects 1 to 12, wherein less than 25% of the disease-modifying osteoarthritis drug in the implant is released from the implant after 15 days in an aqueous environment.
Aspect 14: An implant for intraarticular drug delivery made by the method of any of aspects 1 to 13.
Aspect 15: A method of forming a plurality of microparticles, the method comprising: combining a biodegradable polyester comprising poly(lactic-co-glycolic acid), polycaprolactone, or a combination thereof; and a disease-modifying osteoarthritis drug, in a solvent to provide a first phase; combining the first phase with a second phase comprising an oil to provide an emulsion, wherein the first phase is a minor phase and the second phase is a major phase; and contacting the emulsion with an aqueous environment to precipitate the plurality of microparticles from the emulsion.
Aspect 16: The method of aspect 15, wherein the disease-modifying osteoarthritis drug is punicalagin.
Aspect 17: The method of aspect 15 or 16, wherein the oil comprises sesame oil.
Aspect 18: The method of any of aspects 15 to 17, wherein the biodegradable polyester comprises poly(lactic-co-glycolic acid) having a lactide: glycolide ratio of 25:75 to 75:25.
Aspect 19: The method of any of aspects 15 to 18, wherein the first phase further comprises sucrose acetate isobutyrate.
Aspect 20: The method of any of aspects 15 to 19, wherein the contacting comprises injecting the mixture intraarticularly, and the microparticles are formed in situ in a joint.
Aspect 21: A plurality of microparticles comprising a biodegradable polyester comprising poly(lactic-co-glycolic acid), polycaprolactone, or a combination thereof and a disease-modifying osteoarthritis drug, wherein the plurality of microparticles are made by the method of any of aspects 15 to 20.
Aspect 22: A method of treating osteoarthritis by intraarticular injection of punicalagin.
Aspect 23: The method of aspect 22, wherein the punicalagin is disposed in an implant comprising a biodegradable polyester comprising poly(lactic-co-glycolic acid), polycaprolactone, or a combination thereof for intraarticular drug delivery.
Aspect 24: The method of aspect 22, wherein the punicalagin is disposed in a plurality of microparticles comprising a biodegradable polyester comprising poly(lactic-co-glycolic acid), polycaprolactone, or a combination thereof for intraarticular drug delivery.

1. A method of forming an implant, the method comprising:
providing a mixture comprising
a water-miscible organic solvent;
a water-immiscible organic solvent;
a biodegradable polyester comprising poly(lactic-co-glycolic acid), polycaprolactone, or a combination thereof; and
a disease-modifying osteoarthritis drug; and
contacting the mixture with an aqueous environment to form a precipitate from the mixture to provide the implant.

2. The method of claim 1, wherein
the water-miscible organic solvent comprises N-methyl-2-pyrrolidone, and
the water-immiscible organic solvent comprises benzyl benzoate, benzyl alcohol, or a combination thereof.

3. The method of claim 1, wherein the biodegradable polyester comprises poly(lactic-co-glycolic acid) having a lactide: glycolide ratio of 25:75 to 75:25.

4. The method of claim 1, wherein the mixture comprises N-methyl-2-pyrrolidone, benzyl benzoate, and benzyl alcohol, and a weight ratio of N-methyl-2-pyrrolidone:benzyl benzoate:benzyl alcohol is 10-30:30-50:30-50.

5. The method of claim 3, wherein the poly(lactic-co-glycolic acid) is present in an amount of 15 to 30 weight percent, based on the total weight of the mixture.

6. The method of claim 3, wherein the poly(lactic-co-glycolic acid) is present in an amount of 20 to 25 weight percent, based on the total weight of the mixture.

7. The method of claim 1, wherein the disease-modifying osteoarthritis drug is punicalagin.

8. The method of claim 1, wherein the contacting comprises injecting the mixture intraarticularly, and the implant is formed in situ in a joint.

9. The method of claim 1, wherein less than 25% of the disease-modifying osteoarthritis drug in the implant is released from the implant after 15 days in an aqueous environment.

10. An implant for intraarticular drug delivery made by the method of claim 1.

11. A method of forming a plurality of microparticles, the method comprising:
combining
a biodegradable polyester comprising poly(lactic-co-glycolic acid), polycaprolactone, or a combination thereof; and
a disease-modifying osteoarthritis drug,
in a solvent to provide a first phase;
combining the first phase with a second phase comprising an oil to provide an emulsion, wherein the first phase is a minor phase and the second phase is a major phase; and
contacting the emulsion with an aqueous environment to precipitate the plurality of microparticles from the emulsion.

12. The method of claim 11, wherein the disease-modifying osteoarthritis drug is punicalagin.

13. The method of claim 11, wherein the oil comprises sesame oil.

14. The method of claim 11, wherein the biodegradable polyester comprises poly(lactic-co-glycolic acid) having a lactide:glycolide ratio of 25:75 to 75:25.

15. The method of claim 11, wherein the first phase further comprises sucrose acetate isobutyrate.

16. The method of claim 11, wherein the contacting comprises injecting the mixture intraarticularly, and the microparticles are formed in situ in a joint.

17. A plurality of microparticles comprising a biodegradable polyester comprising poly(lactic-co-glycolic acid), polycaprolactone, or a combination thereof, and a disease-modifying osteoarthritis drug, wherein the plurality of microparticles are made by the method of claim 11.

18. A method of treating osteoarthritis by intraarticular injection of punicalagin.

19. The method of claim 18, wherein the punicalagin is disposed in an implant comprising a biodegradable polyester comprising poly(lactic-co-glycolic acid), polycaprolactone, or a combination thereof for intraarticular drug delivery.

20. The method of claim 18, wherein the punicalagin is disposed in a plurality of microparticles comprising a biodegradable polyester comprising poly(lactic-co-glycolic acid), polycaprolactone, or a combination thereof for intraarticular drug delivery.
